(19) United States
(12) Patent Application Publication
(10) Pub. No.: US 2011/0054235 A1
(43) Pub. Date: Mar. 3, 2011

(54) PACKAGING SYSTEM FOR BRACHYTHERAPY IMPLANT AND CUTTING THEREOF

(76) Inventors: Christopher D. Drobnik, Wauconda, IL (US); Michael W. Drobnik, Downers Grove, IL (US); James Stewart, Mooresville, NC (US); Michael Johnson, Charlotte, NC (US); Mark Ralph, Somerset, WI (US)

(21) Appl. No.: 12/733,097

(22) PCT Filed: Aug. 22, 2008
(86) PCT No.: PCT/US2008/010011

§ 371 Date: Jun. 1, 2010

Related U.S. Application Data
(60) Provisional application No. 61/015,774, filed on Dec. 21, 2007.

(30) Foreign Application Priority Data
Aug. 24, 2007 (US) US2007/007673

(51) Int. Cl. A61M 36/00

(52) U.S. Cl. 600/8

(57) ABSTRACT

The present disclosure relates to a thermo formed tray having an upper half and a lower half. The upper half and the lower half define a closed configuration when the upper half and the lower half are substantially adjacent one another and an open configuration when the upper half and the lower half are substantially apart from one another. A predetermined area is provided in at least one of the upper half and the lower half for containing a product in the closed configuration, the product having at least one radioactive seed held in a predetermined seed configuration. At least one cutter guide is contained in the thermo formed tray.

This application claims benefit of priority to U.S. Provisional Application No. 61/015,774, filed Dec. 21, 2007, and entitled “Packaging System for Brachytherapy Implant” (the disclosure of which is incorporated herein by reference in its entirety).

This application claims benefit of priority to International Application No. PCT/US2007/076736, filed Aug. 24, 2007, and entitled “Therapeutic and Directionally Dosed Implants” (the disclosure of which is incorporated herein by reference in its entirety) which claims priority to U.S. Provisional Application No. 61/823,555, filed Aug. 25, 2006.

### BACKGROUND

Bodily cancers are commonly treated using radiation therapy. Radiation therapy employs high energy radiation to kill cancer cells. One type of radiation therapy is brachytherapy, in which a source of radiation is in direct contact with an afflicted tissue. A common brachythcrapy treatment, transperincal seed implantation, involves placing radioactive seeds in the prostate gland to kill prostate gland cancer cells. A physician employs tools such as ultrasound, computerized tomography (“CT”) scans, and X-ray images in concert with dose-planning computer software programs to evaluate the medical condition of a patient. The physician constructs an optimal treatment plan to evenly distribute radiation throughout the afflicted tissue. Radioactive seeds of discrete radioactive strengths are inserted through multiple implantation needles at positions in the prostate gland corresponding to the treatment plan. Multiple implantation needles are required to insert the radioactive seeds into multiple locations in the afflicted tissue, with each needle containing a specified arrangement of the radioactive seeds.

Although brachytherapy is perhaps most often effected by needle implantation, other implantation methods have also been used. One example is a configuration employing seeds and sutures. To make such a configuration, the physicians have utilized a seed product (e.g., Seed in Carrier, manufactured by Oncura) that consists of seeds disposed within a suture material. The sutures are weaved through a piece of bioabsorbable fabric to yield a planar array of seeds. This array is then used to irradiate a tumor bed, most commonly following a lung resection, by sewing the array to the wall of the tumor bed.

Gross surgical removal of tumorous tissue can leave behind traces of cancerous tissue, which can result in metastasis, or recurrence of the tumor. Thus, the site of removal of a tumor is often treated postoperatively in an attempt to destroy any such diseased tissue left behind by the surgery. Conventional techniques for treating the site of surgical removal of a tumor include post-operative administration of radiation, chemotherapy, and/or heat. Another method is disclosed in U.S. Pat. No. 5,030,195, the disclosure of which is incorporated by reference herein. In accordance with that disclosure, seeds can be threaded into a mesh, and the mesh embedded in a nonabsorbable silicone compound. Once the exact location and extent of a tumor is determined, the tumor is removed, and the mesh/silicone material is embedded in a region where residual tumor cells may exist.

Proper seed placement and seed retention at the implantation site strongly influence the success or failure of a brachytherapy procedure. As described above, seed implantation devices may contain a plurality of seeds that may be separated by spacers. Prior implantation devices and methods do not reliably maintain proper seed spacing during and after implantation. Therefore, a device and/or method of reliably maintaining proper seed spacing during and after implantation would be of great benefit to brachytherapy patients.

Loose seeds implanted in the prostate, especially those that arc extra-capsular (located outside the capsule of the prostate), may possibly migrate within the patient. Because extra-capsular tissue is less dense than tissue within the capsule of, e.g., the prostate, prior brachytherapy seed implantation devices and methods cannot effectively maintain the location of seeds in the extra-capsular material. These seeds may migrate and fail to provide radiation where needed. Migrating radioactive seeds not only fail to provide needed radiation therapy at the treatment site, but also may cause damage to other radiation-sensitive areas of the body. Therefore, a device and/or method of preventing migration of radioactive seeds in tissues and/or fluids of varying densities and consistencies would be of great benefit to brachythcrapy patients.

In view of the above, it would be desirable to have an implant, whether standard or custom, which is capable of delivering radiation to a patent in need thereof without the above-mentioned disadvantages. The present disclosure provides the ability to present the seeds to the physician ready-made on a sheet of material, rather than only supplying them loose or pre-made into simple “lines.” This can provide a more usable product that is more amenable to placement on the exterior portion of a tumor, along the suture line of a resected tumor, as well as enabling placement of seeds within the “hole” created by the excision of a tumor in order to better treat the microscopic disease located in the non-excised tumor margins. The present disclosure provides several innovations that can be incorporated to allow ease of manufacture, ease of handling, flexibility of deployment, dose delivery and reliability, etc.

### SUMMARY

According to one aspect of the present disclosure, there is provided an implant comprising at least one sheet of a biocompatible material, at least one shielding apparatus fixed to the biological material, and at least one radioactive seed partially disposed in the shielding apparatus.

According to another aspect of the present disclosure, there is provided a method for treating a patient, comprising fixing to the tissue of the patient at least one implant comprising at least two sheets of a biocompatible material, and at least one radioactive seed disposed between said sheets of material.

According to yet another aspect of the present disclosure, there is provided a method for treating a patient, comprising surgically excising at least a portion of a tumor from surrounding tissue, and providing at least one implant at the locus of said surrounding tissue, wherein the implant comprises at least two sheets of a biocompatible material, and at least one radioactive seed disposed between said sheets of material.

According to another aspect of the present disclosure, there is provided a thermoformed tray having an upper half and a lower half. The upper half and the lower half define a closed configuration when the upper half and the lower half are substantially adjacent one another and an open configuration when the upper half and the lower half are substantially apart from one another. A predetermined area is provided in at least one of the upper half and the lower half for containing a product in the closed configuration, the product having at least one radioactive seed held in a predetermined seed configuration. At least one cutter guide is contained in the thermoformed tray.

According to yet another aspect of the present disclosure, there is provided a thermoformed tray having an upper half and a lower half. The upper half and the lower half defining a closed configuration when the upper half and the lower half are substantially adjacent one another and an open configuration when the upper half and the lower half are substantially apart from one another. A predetermined area in at least one of the upper half and the lower half contains a product in the closed configuration, the product having at least one radioactive seed held in a predetermined seed configuration. A cutting instrument and a cutter guide are also contained in the thermoformed tray. A first area in at least one of the upper half and the lower half holds the cutter guide, and a second area in at least one of the upper half and the lower half holds the cutting instrument.

### BRIEF DESCRIPTION OF THE DRAWINGS

The disclosed embodiments can be better understood with reference to the following drawing. The components in the drawing arc not necessarily to scale.

FIG. 1, according to various embodiments, is an illustration of an implant in accordance with the present disclosure.

FIG. 2, according to various embodiments, is a detailed illustration of an implant having radioactive seeds in accordance with the present disclosure.

FIG. 3, according to various embodiments, is an overall illustration of an implant having radioactive seeds in accordance with the present disclosure.

FIG. 4, according to various embodiments, is a detailed illustration of an alternative embodiment of an implant having radioactive seeds in accordance with the present disclosure.

FIG. 5, according to various embodiments, is an overall illustration of an alternative embodiment of an implant having radioactive seeds in accordance with the present disclosure.

FIG. 6, according to various embodiments, is an overall illustration of an implant having radioactive seeds in a predetermined pattern in accordance with the present disclosure.

FIG. 7, according to various embodiments, is an overall illustration of an implant having radioactive seeds in another predetermined pattern in accordance with the present disclosure.

FIG. 8, according to various embodiments, is an overall illustration of an implant having radioactive seeds disposed in a shielding material that is attached to a sheet of biocompatible material.

FIG. 9, one embodiment of the present method and apparatus may also have an outer shielding case that encases the upper and lower halves of a thermoformed tray.

FIG. 10 is an exploded perspective view of a thermoformed tray 1000 according to the present method and apparatus.

FIG. 11 depicts the cutter guide located over a bioabsorbable felt.

FIG. 12 depicts an embodiment of the product as a bioabsorbable felt 1202 containing radioactive seeds.

### DESCRIPTION

According to various embodiments, the present disclosure relates to an implant comprising at least one seed in a carrier. For example, the implant comprises a plurality of seeds. The implant can comprise an array, for example a planar array, of seeds. The seeds can be disposed in an array on the material, based on horizontal and vertical separation of the seeds. The implant can also comprise seeds arranged in a three dimensional construction. For example, the seeds can be disposed in a flexible mass (such as a sphere) of mesh that could be collapsed, inserted into a body cavity and allowed to naturally expand to fill the cavity. According to various embodiments, the mass could be flexible enough to conform to an area that would not be perfectly spherical. According to various embodiments, the mass could be expanded and/or compressed to a shape by natural stresses or forces.

According to various embodiments of the disclosure, the array can be provided pre-made, or standardized, with definite spacing between the seeds. This known array allows calculation of dosimetry to the treated area. The array could be constructed with a standard spacing, or be customized to a seed pattern desired by the end user. For example, the carrier could be provided without radioactive seeds and with provisions in the carrier, such as pouches or slits, to allow for loading of individual seeds by the end user. In this manner, the seeds can be disposed in the carrier with cither standardized or customized spacing. A discrete seed spacing could be advantageous in that the end user would not have to weave a suture containing seeds into a mesh. This could provide a time savings, and could ensure that the seeds would have a definite spacing (independent of the skill of the end user in weaving a filament of seeds) and provide reproducible and calculable dosimetry.

According to various embodiments, the implant comprises a bioabsorbable or permanent carrier. Alternatively, the implant can comprise both bioabsorbable and permanent components in the carrier. The use of either a bioabsorbable or permanent carrier allows the physician to tailor the mechanical properties of the implant to fit the tumor type or location of the tumor/tumor bed.

There are a variety of radioactive seeds that can be used in accordance with the present disclosure. Suitable non-limiting examples of such seeds include, for example, I¹²⁵, Pd¹⁰³, Cs¹³¹, Au¹⁹⁸, Co⁶⁰, and Ir¹⁹². Those of ordinary skill in the a appreciate that any seed or radioactive particle capable of providing a therapeutic dose of radiation can be used. Seeds can be made of a number of different materials known to the ordinary practitioner. For example, the seeds can be in the form of a metallic capsule, a polymer, a ceramic, a ribbon, or can be particulate in nature. Any form capable of providing the desired dose of radiation can be used.

The implant can comprise a variety of materials (in addition to the seeds). For example, the seeds can be entrained within a sheet, woven mesh, knitted mesh, felt, polymeric sheet, fabric, etc. According to various embodiments, the seeds arc entrained within a non-absorbable mesh. Suitable non-absorbable meshes arc well-known, and include those disclosed in, for example, U.S. Pat. No. 6,971,252 (the disclosure of which is incorporated by reference herein). The meshes can be constructed of at least one of polypropylene, polyester, polyurethane, stainless steel, titanium, carbon fiber, nitinol, and other materials. According to various embodiments, the seeds can be entrained in a nonabsorbable material, such as a non-absorbable polymeric sheet. Suitable non-limiting examples of polymeric sheets include polyurethane and silicone.

According to various embodiments, the seeds can be entrained within an absorbable mesh, felt, or sheet. Absorbable materials arc well-known to those of ordinary skill in the art, and can be constructed of, for example, polydioxanone, polylactide, polyglycolic acid, absorbable polyurethane, and collagen.

The seeds can be disposed in the carrier via a number of different mechanisms. For example, the seeds can be attached to the carrier via adhesives, welding, thermal bonding, sewing, entrainment between two sheets of material, or placement into formed pockets on the material. The entrainment between two sheets of material can be accomplished by heat staking around the seeds to affix the two sheets together. The heat staking technique could be advantageous in that a second chemical and/or material would not necessarily need to be added to affix the seeds, and the tedious task of sewing with radioactive materials could be avoided. The original properties of the entrainment material, most importantly the ability of tissue to grow into the pores of the material, could also be maintained with minimal disruption during the staking process. The seeds can also be affixed to the material by placing the seeds within a holder or shielding apparatus having a feature that mates with a corresponding feature in, or on the opposite side of, the material.

According to various embodiments, the carrier materials can be homogeneous, or can be constructed of layers or areas of dissimilar materials (e.g., a polypropylene mesh welded to a polyester mesh, with the seeds trapped between). A suitable construction can be selected to adjust physical and performance qualities, including but not limited to flexibility, degree of tissue in-growth, tensile or flexural strength, avenues for sterilization or processing, degree of seed retention, visibility by medical imaging modalities, attachment method to tissue or bone, degradation time, adhesion (e.g., provide a fast-absorbing collagen layer on the outside of a slow-absorbing polylactide material) and control of tissue erosion.

The carrier material can incorporate additional elements for a variety of purposes. Suitable non-limiting examples of such elements include fiducial markers for visualization/localizing by medical imaging modalities (ultrasound, fluoroscopy, MRI, CT, etc.); visual markings indicating alignment, seed placement, seed placement distances, and/or tissue attachment points; features to ease assembly and alignment such as “corduroy” type grooves, dimples, and formed depressions; and coatings to increase/reduce adhesion, promote/retard in-growth, cause coagulation of blood, provide tumoricidal activity, increase biocompatibility, reduce microbiological growth, reduce inflammation, deliver analgesics, etc. In addition, the carrier material can have incorporated therein, or attached thereon, features to case attachment to tissue such as, for example, loops, arms, filaments, sutures, and staples.

According to various embodiments, the implant can comprise a radiation shielding backing material to afford a directional radiation dose. Suitable non-limiting examples of such materials include bismuth- or barium-loaded polymers. This backing material can be in the form of a solid sheet, or have open areas to allow selective dose transmission. Such a backing material could be useful to direct the dose towards areas of interest while shielding healthy or sensitive tissues or organs. The backing material can be permanent (e.g., bismuth-loaded silicone), removable or bioabsorbable.

Radioactive seeds, such as the BrachySource seeds sold by Bard Brachytherapy (Carol Stream, Ill.) provide a symmetrical “4π” does distribution around the seed. Such seeds can be useful for treating various types of tumors such as prostate tumors, where a tumoricidal dose is desired all the way around a seed.

There are various clinical situations where a limited directional dose or field is desired. For example, to implant seeds in an excised tumor bed following a radical prostatectomy, the clinician would want to irradiate the tumor bed but may not want to irradiate the rectum or bladder. With known seed designs and deployment, this could be difficult because seeds are typically free to rotate or shift from their implanted position. If the seeds were attached to a mesh as described above, the seeds would be fixed and the placement of the mesh would necessarily fix the placement and orientation of the seeds. The radiation field of the seed could then be altered to provide a uni-directional dose.

According to various embodiments, there can be advantages associated with modifying the radiation field surrounding the seed without modifying the seed itself. For example, the modification could potentially be used with any seed in the marketplace; a seed manufacturer would not have to keep as extensive an inventory of different seeds; and the proven structural integrity of a given seed would be unchanged. Several different dose profiles could be offered by merely modifying the exterior shielding apparatus. The shielding method could aid in fixing the seed in the body to confirm the direction of the dose in relation to body structures. In addition, the shielding apparatus could offer a visual verification of the dose direction (which wouldn't necessarily be available with an internal dose modification).

According to various embodiments, there is provided herein radioactive seeds that incorporate a shielding component in one or more desired directions. The shielding can be provided by any biocompatible material such as stainless steel, titanium, tungsten, gold, platinum, etc., with the exact material being selected based on the desired degree of shielding.

The shielding material can be homogenous, or can comprise a plurality of layers (e.g., spring steel electroplated with gold). The layers can be selected in order to modify at least one of biocompatibility, manufacturability, cost, radiopacity, susceptibility to galvanic corrosion, functionality, and durability.

According to various embodiments, the shielding apparatus can be fixed to the seed as a flat plate or as a conformal structure (e.g., a piece of foil could surround a portion of the diameter of the seed). The shielding apparatus can be attached to the seed by any suitable attachment method such as, for example, adhesive, welding (laser, resistance, etc.), soldering, mechanical entrapment, electroplating, etc. According to various embodiments, mechanical entrapment can be accomplished by at least one of a feature that allows a seed to be “snapped” in place; a feature that has seed placement in the shielding followed by crimping, forming, etc., to complete the entrapment; a feature that naturally confirms around the seed at body temperature (e.g., construct the shielding material from nitinol, which is a flat sheet at cool temperatures but which wraps around the seed when warmed to room or body temperature).

The shielding apparatus can include at least one feature that prevents, or at least reduces, rotation or movement of the seed relative to the mesh. For example, the shielding material can have flat areas extending from the seed assembly. The material can comprise barbs or rough areas that grip the mesh structure. According to various embodiments, if the seeds were sewn in place, the shielding material could be provided with ridges, holes, or other features capable of securing the thread.

According to various embodiments, the shielding apparatus could be provided with various agents such as, for example, agents that modify adhesion, promote or retard ingrowth.

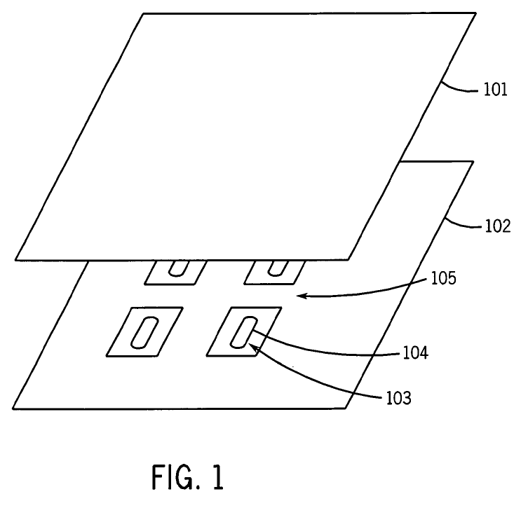

The above-described features of the various embodiments of the present method and apparatus are depicted in FIGS. 1-8.

FIG. 1, according to various embodiments, is an illustration of an implant in accordance with the present disclosure. In this embodiment a first sheet of material 101 may be operatively coupled to a second sheet of material 102. The second sheet of material 102 may have formed pockets 103 which hold seeds 103. The first and second sheets of material 101, 102 may be adhered to one another by heat staking around the seeds in areas 105.

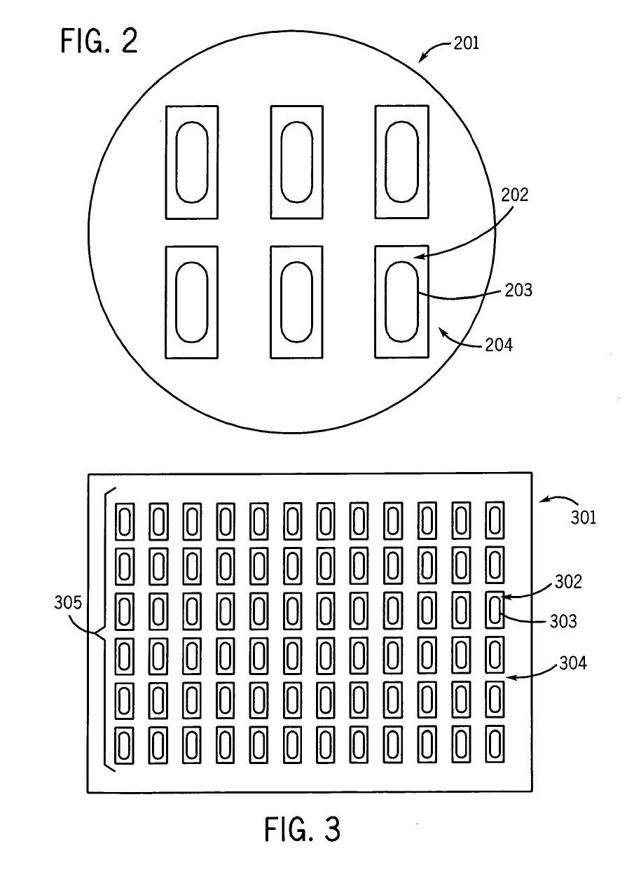

FIG. 2, according to various embodiments, is a detailed illustration of an implant 201 having radioactive seeds 203 in accordance with the present disclosure. The seeds 203 may be held in pockets 202 by staking areas 204. In this embodiment each pocket 202 may be surrounded by one common staking area 204.

FIG. 3, according to various embodiments, is an ove[tau]all illustration of an implant 301 having radioactive seeds 303 in accordance with the present disclosure. The seeds 303 may be held in pockets 302 by staking area 304. The seeds 303 in the pockets 302 may be arranged in a predetermined pattern 305. The pattern 305 may be constructed with a standard spacing as depicted, or may be customized to a seed pattern desired by the end user.

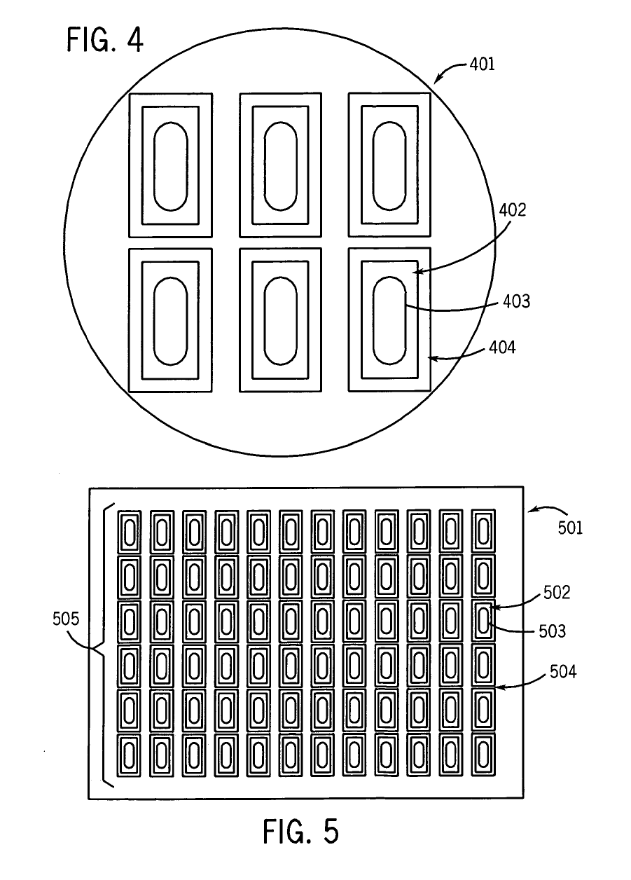

FIG. 4, according to various embodiments, is a detailed illustration of an alternative embodiment of an implant 401 having radioactive seeds 403 in accordance with the present disclosure. The seeds 403 may be held in pockets 402 by staking areas 404. In this embodiment each pocket 402 is surrounded by its own respective staking area 404.

FIG. 5, according to various embodiments, is an overall illustration of an alternative embodiment of an implant 501 having radioactive seeds 503 in accordance with the present disclosure. The seeds 503 may be held in pockets 502 by individual staking areas 504. The seeds 503 in the pockets 502 may be arranged in a predetermined pattern 505. The pattern 505 may be constructed with a standard spacing as depicted, or may be customized to a seed pattern desired by the end user.

FIG. 6, according to various embodiments, is an overall illustration of an implant 601 having radioactive seeds 603 in a predetermined pattern 605 in accordance with the present disclosure. The seeds 603 may be held in pockets 602 by individual staking areas 604. The implant 601 may incorporate additional elements for a variety of purposes, such as, fiducial markers 606, visual markings 607, coatings 608, and attachment elements 609.

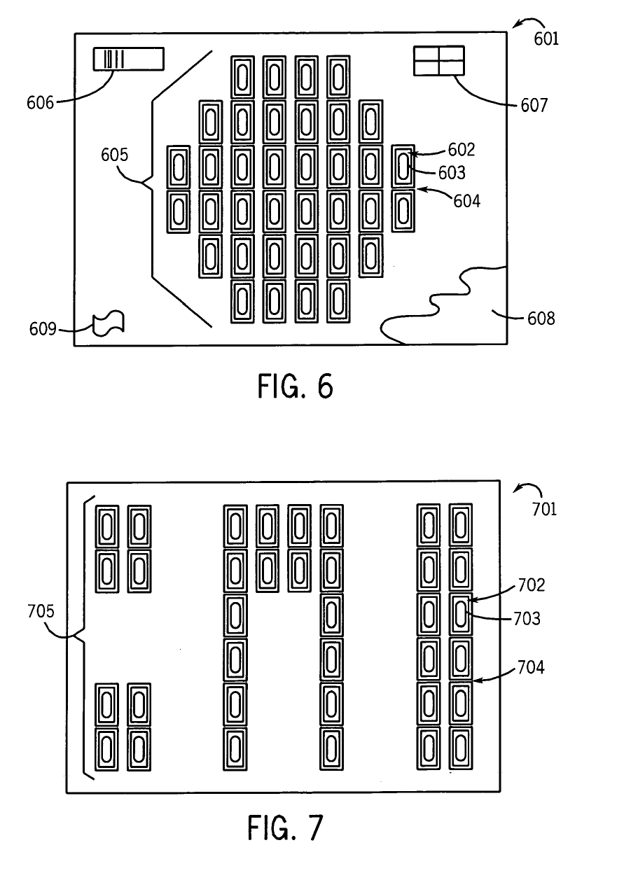

FIG. 7, according to various embodiments, is an overall illustration of an implant 701 having radioactive seeds 703 in another predetermined pattern 705 in accordance with the present disclosure. The seeds 703 may be held in pockets 702 by individual staking areas 704.

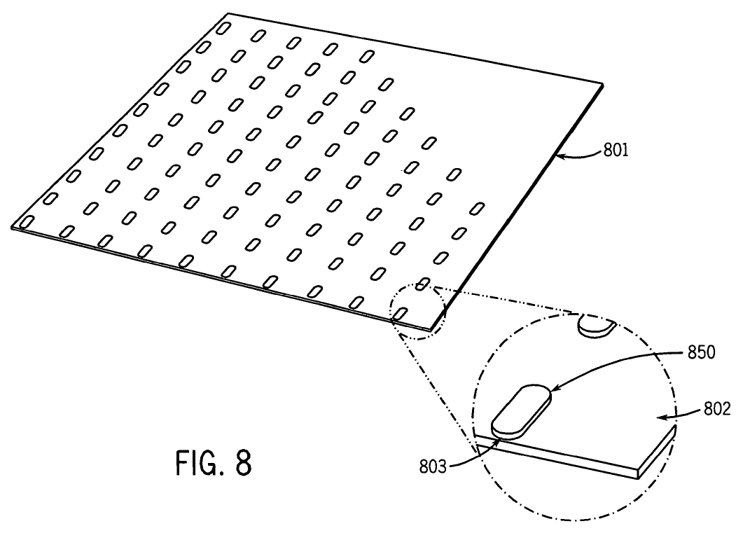

FIG. 8, according to various embodiments, is an overall illustration of an implant 801 having radioactive seeds 803 in a predetermined pattern in accordance with the present disclosure. The seeds 803 arc partially disposed in shielding apparatus 850. Shielding apparatus 850 is attached to biocompatible material 802.

The implants can be attached to tissue using a variety of different methods. For example, the implants can be affixed to tissue via at least one of sutures, staples, tacks, adhesives, physical entrapment (chevrons), or other standard tissue-anchoring means. The implants can be permanently flexible, or can be rigid and formed into particular rigid shapes using heat and/or pressure based on the particular application. The implants could be constructed of materials that would change physical properties when contacted with body fluids or exposed to body temperature.

According to various embodiments, the implants can be applied externally or internally. For example, the implants can be inserted laproscopically or by open surgery. The implants can be used in the body, or externally (i.e., a skin patch). The implant can be inserted into a tumor bed. For example, a tumor can be excised from a body cavity and the implant can be fixed to the locus thereof. The implant can be fixed to the tumor bed by a variety of different methods, including suturing, stapling, and adhesion. In the case of a spherical or semispherical implant, the implant can be inserted into the cavity and permitted to expand, thereby filling at least a portion of the cavity.

The general packaging concept is for a bioabsorbable felt, for example, to be contained within a two-component thermoformed tray, held within a flat pewter tray or case with a telescoping lid. The pewter tray may be contained in a thermoform tray with a Tyvek lid as the sterile barrier. This lidded tray may be held within a paperboard carton, with the carton held in a die cut foam insert that goes inside an outer corrugate shipping box. The foam shipping insert may have cavities for holding additional loose seeds, calibrated seeds, seeds in Mick cartridges and/or other non-radioactive components commonly used in the surgical procedure (e.g. to make the box a surgical kit for a single patient).

The thermoformed tray may have cut out sections to allow on board storage of a scalpel to cut the mesh, and a cutter guide. The tray may have an upper and lower half that are within a telescoping pewter tray that is sealed with shrink wrap. The shrink wrap helps hold the assembly together under tension, limiting movement of the contained components without resorting to a difficult-to-open heat seal or something equivalent.

The thermoformed tray may have: alpha and numeric characters printed on the x- and y-axes to mimic typically nomenclature in use in brachytherapy to help identify seed placement for dose planning purposes; a recessed grid pattern on the lower tray to aid in cutting the material in a straight line; and may be constructed of clear materials, so the tray can be placed on a light box or similar. This will allow light to pass through the assembled product, with the resulting “shadows” of seeds being used as a secondary quality inspection technique by the end user to verify seeds are in the correct area. This may be done while the upper and lower tray components are still in place, minimizing the potential for contamination of the sterile product.

The thermoformed tray may also have a clear, recessed upper portion that is the same size as the product itself. This upper portion may be used to contain documentation as to the loading plan of the product below (e.g. autoradiograph, digital image, dose plan, etc.). This documentation may be printed onto clear stock (e.g. transparency sheets) so that by back lighting the product so that the end user can “look through” the documentation and product itself to verify everything “lines up” as specified. The upper recessed portion may hold a piece of regular or Polaroid-type film to allow a radiograph to be taken by the end user.

The lower tray may have alignment notches or features around the inside perimeter to aid in cutting the product as desired (used in conjunction with the “cutter guide” to be discussed below). The lower tray may have upward-facing barbs or protrusions to help hold the mesh in place during transport, handling, cutting, etc. The recessed area in the lower tray may be used as a bath to soak the product prior to placement in the body. The soaking material may be saline (to increase flexibility), a chemotherapeutic agent, a coagulant or tissue glue (to help hold the product in place after initial placement), an anesthetic, and anti-inflammatory agent, etc.

The upper tray may have finger holes, tabs, etc. to facilitate removal of the tray lid. Four outer recessed areas may be provided in the corners of lower tray to provide “finger notches” or areas that allow the end user to easily grasp the mesh for removal from the tray.

A cutter guide may be a plastic or metal formed part to help the end user cut the product in the desired shape (if cutting is necessary based on the exact use of the product). The cutter guide may have the following attributes: made of metal or other radiation-shielding material to reduce radiation exposure to the operator; made of a clear material to allow the product underneath to be seen, minimizing the risk of cutting a seed, cutting in the wrong place, etc.; have a measurement rule or other markings to help the operator determine dimensions of the product; have features on the underside that can match features on the tray to ensure that the guide lines up where intended (e.g. so the cutter guide can only be placed where it is impossible to cut a seed); have a central groove or pocket running the length of the device to receive a scalpel blade to ensure the scalpel goes in a straight line; be a simple straight edge, where the operator runs the blade along the edge of the device to make the cut; be of a sufficient width such that when held by the operator, the blade path is far removed from the fingers so that risk of injury is minimized; have features on the underside like cavities or troughs that cover the seeds below, minimizing the potential for blade-seed interaction and subsequent damage; a built-in cutter that can be moved along the guide to cut the felt (like the new style saran wrap cutter); a roughened or barbed lower surface to better grip the felt while cutting; a roughened upper surface to minimize slippage when used with gloved hands in the sterile environment.

The product, such as a bioabsorbable felt may have one or more of the following features: tabs or elements on the perimeter that help align and stabilize the product within the package; printing that would aid in dose planning, seed location, etc., which may be alpha-numeric characters to mimic typically brachytherapy dose planning conventions; position of the seeds in the mesh marked following loading, as a further visual indication of seed presence; markings or printing being radiopaque to aid in localization using fluoroscopy.

In addition to perimeter markings, the mesh may be marked internally with a pattern (e.g. grid) to help manufacturing personnel accurately place seeds in the correct areas prior to final product assembly. These lines may also be used by the end user as “cutting lines”.

Markings on the exterior of the mesh may be placed on only one side of the mesh to be used as a visual indicator (e.g. if the seed product is used with a directional radiation shield, the markings may be used to delineate the “hot” side and “cold” side). The markings may also be used to provide a visual correlation between provided quality assurance documentation and product construction (e.g. a digital image may be taken of the felt and the digital image may be married up with an autoradiograph so that the end user can know the correct orientation of seeds in the product).

Thus, in general an embodiment of the present method and apparatus may have a thermoformed tray having an upper half and a lower half, a predetermined area in at least one of the upper half and the lower half for containing a product, and at least one cutter guide. The product may be, for example, a bioabsorbable felt containing radioactive seeds or a mesh containing radioactive seeds.

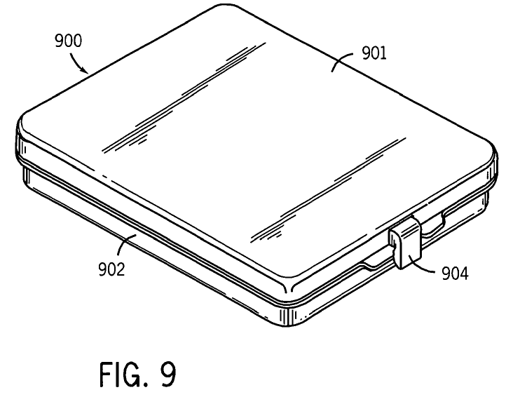

As depicted in FIG. 9, one embodiment of the present method and apparatus may also have an outer shielding case 900 that encases the upper and lower halves of the thermoformed tray. The outer shielding case 900 may have a variety of different forms, for example, the outer shielding case 900 may be a telescoping case having an upper section 901 and a lower section 902. The outer shielding case 900 may optionally have a latch 904, or other type of structure that securing mechanism. The outer shielding case 900 may be formed of at least one of lead, stainless steel, bismuth- or tungsten-loaded plastic, and pewter. Additionally, the outer shielding case 900 may be sealed with a shrink-wrap material (not shown).

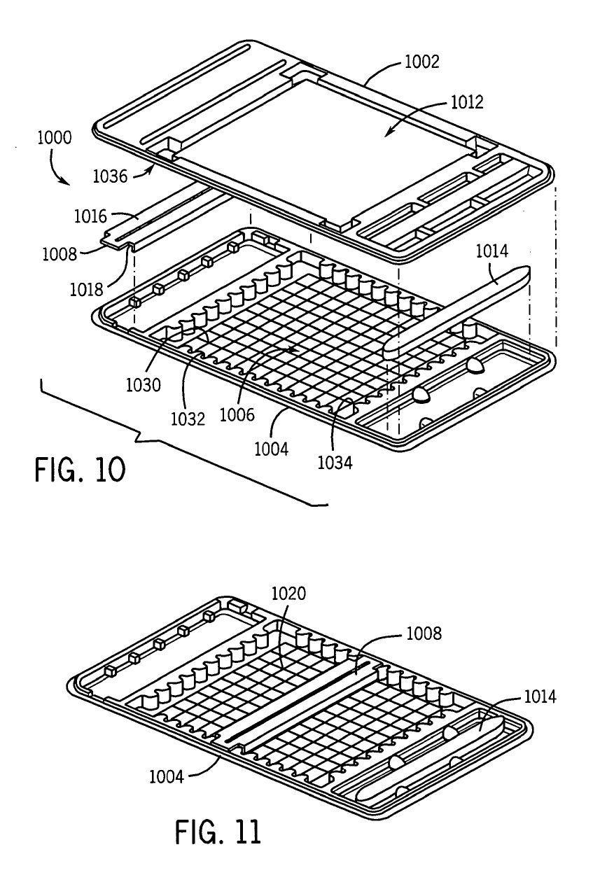

FIG. 10 is an exploded perspective view of a thermoformed tray 1000 according to the present method and apparatus. The thermoformed tray 1000 may have an upper half 1002 and a lower half 1004. The upper half 1002 and the lower half 1004 may define a closed configuration when the upper half 1002 and the lower half 1004 are substantially adjacent one another, and an open configuration when the upper half 1002 and the lower half 1004 are substantially apart from one another.

At least one of the upper half 1002 and the lower half 1004 may have a predetermined area 1006 for containing a product in the closed configuration, the product having at least one radioactive seed held in a predetermined seed configuration. The product may be, for example, a bioabsorbable felt. At least one of the upper half 1002 and the lower half 1004 of the thermoformed tray 1000 may be formed of a transparent material. In addition, the thermoformed tray 1000 may also have at least one of alpha characters and numeric characters 1010 that are printed on x and y axes to mimic brachytherapy nomenclature to help identify seed placement for dose planning.

The thermoformed tray 1000 may also have at least one cutter guide 1008. The cutter guide 1008 may have a roughened lower surface 1018 to better grip the product while cutting, and a roughened upper surface 1016 to minimize slippage. Alternatively, the cutter guide 1008 may have a built-in cutter that is moveable along the guide to cut the product.

Furthermore, the lower half 1004 of the thermoformed tray 1000 may be structured to function as a bath for containing a soaking solution for soaking the product prior to placement in a body. For example, an area for the bath may also be the predetermined area 1006. The soaking material may be, for example, one of a saline, a chemotherapeutic agent, a coagulant or tissue glue, an anesthetic, and anti-inflammatory agent.

In an alternative embodiment the upper portion 1002 may have a clear, recessed area 1012 that is approximately a same size as the product. The recessed area 1012 may contain documentation as to the loading plan of the product. The documentation may be printed on clear stock so that by back lighting the product an end user is able to look through the documentation and product for verification.

The cutter guide 1008 may be formed of a radiation-shielding material to reduce radiation exposure from the product. Also, the cutter guide may have measurement markings to help an operator determine dimensions of the product.

FIG. 11 depicts the cutter guide 1008 located over a bioabsorbable felt 1020. The cutter guide 1008 is in position so that the bioabsorbable felt 1020 may be cut by the cutting instrument 1014.

The thermoformed tray 1000 may also have a cutting instrument 1014. The cutter guide 1008 may have a holding area of such width that, when an operator holds the cutter guide 1008, a blade path of the cutting instrument 1014 is spaced from fingers of the operator so that risk of injury is minimized.

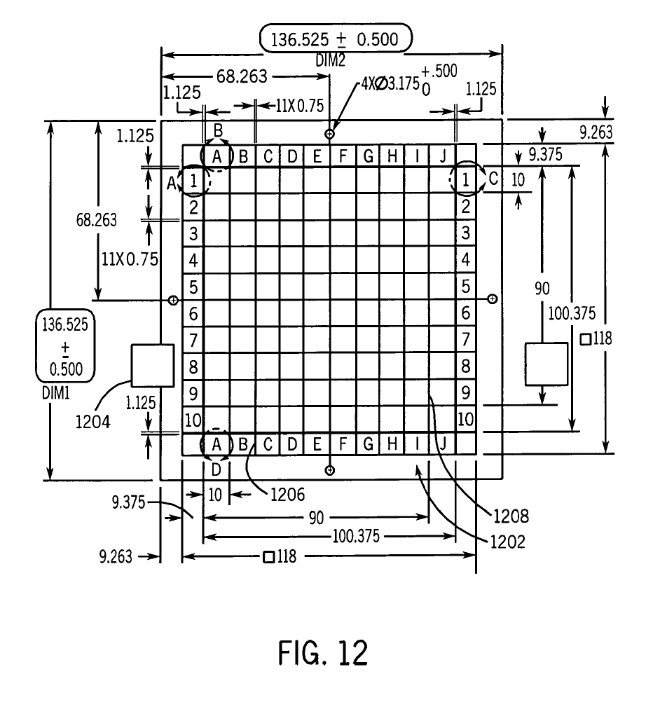

FIG. 12 depicts an embodiment of the product as a bioabsorbable felt 1202 containing radioactive seeds in a seed placement grid 1208. Positions of the seeds in the bioabsorbable felt 1202 are marked following loading, as a further visual indication of seed presence. The product has tab elements 1204 on a perimeter of the bioabsorbable felt 1202 that help align and stabilize the bioabsorbable felt 1202 within the upper half and the lower half of the thermoformed tray. Also, the bioabsorbable felt 1202 may have printing 1206 that aids in at least dose planning and seed location. The printing 1206 may comprise alphanumeric characters to mimic typically brachytherapy dose planning conventions.

In a further embodiment the printing 1206 may be radiopaque to aid in localization using fluoroscopy. In another embodiment the bioabsorbable felt 1202 may have perimeter markings, and the bioabsorbable felt 1202 may be marked internally with a pattern to help manufacturing personnel accurately place seeds in correct areas prior to final product assembly. The pattern may be used as cutting lines.

In another embodiment perimeter markings may be located on only one side of the bioabsorbable felt 1202 and may be used as a visual indicator. The perimeter markings may also be provided as a visual correlation between quality assurance documentation and product construction.

In an alternative embodiment of the present method and apparatus the lower half 1004 of the thermoformed tray 1000 may have a recessed grid pattern 1030 to aid in cutting the bioabsorbable felt 1202 in substantially a straight line. Furthermore, the lower half 1004 may have alignment structures 1032 around an inside perimeter of the lower half 1004 to aid in cutting the bioabsorbable felt 1202. The lower half 1004 may also have upward-facing protrusions 1034 to hold the bioabsorbable felt 1202 in place during transport, handling, and cutting.

The upper half 1002 of the thermoformed tray 1000 may have finger engagement structures 1036 to facilitate removal of the upper half 1002. The lower half 1004 may also have finger engagement structures 1038. The finger engagement structures 1038 may be recessed areas in corners of the lower half 1004 to provide areas that allow a user to easily grasp the bioabsorbable felt 1202 for removal from the lower half 1004.

In one embodiment the cutter guide 1008 may be made of a clear material to allow the bioabsorbable felt 1202 underneath to be seen, minimizing risk of incorrect cutting of the bioabsorbable felt 1202. Also, the cutter guide 1008 may be structured on an underside thereof to match structural features on the lower half 1004 to ensure that the cutter guide 1008 is locatable in the lower half 1004 only where it is impossible to cut a seed in the bioabsorbable felt 1202. In an alternative embodiment the cutter guide 1008 may have one of a central groove and pocket running a length of the cutter guide 1008 to receive the cutting instrument 1014 to ensure cuts in the bioabsorbable felt 1202 by the cutting instrument 1014 are in a substantially straight line. Furthermore, the cutter guide 1008 may have structural elements on an underside thereof that cover seeds of the bioabsorbable felt 1202, minimizing a potential for interaction between the cutting instrument 1014 and the seeds and subsequent damage to the seeds.

For the purposes of this specification and appended claims, unless otherwise indicated, all numbers used in the specification and claims arc to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention arc approximations, the numerical values set forth in the specific examples arc reported as precisely as possible. Any numerical value, however, inherently contains certain errors necessarily resulting from the standard deviation found in their respective testing measurements. Moreover, all ranges disclosed herein arc to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a mesh” includes two or more meshes.

Other various embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An apparatus, comprising:
thermoformed tray having an upper half and a lower half, the upper half and the lower half defining a closed configuration when the upper half and the lower half are substantially adjacent one another and an open configuration when the upper half and the lower half are substantially apart from one another;
a predetermined area in at least one of the upper half and the lower half for containing a product in the closed configuration, the product having at least one radioactive seed held in a predetermined seed configuration;
a cutting instrument and a cutter guide;
a first area in at least one of the upper half and the lower half for holding the cutter guide; and
a second area in at least one of the upper half and the lower half for holding the cutting instrument.

2. The apparatus of claim 1, wherein the lower half has a recessed grid pattern to aid in cutting the product in substantially a straight line.

3. The apparatus of claim 1, wherein the lower half has alignment structures around an inside perimeter of the lower half to aid in cutting the product.

4. The apparatus of claim 1, wherein the lower half has upward-facing protrusions to hold the product in place during transport, handling, and cutting.

5. The apparatus of claim 1, wherein the upper half has finger engagement structures to facilitate removal of the upper half.

6. The apparatus of claim 1, wherein the lower half has finger engagement structures, and wherein the finger engagement structures are recessed areas in corners of the lower half to provide areas that allow a user to easily grasp the product for removal from the lower half.

7. The apparatus of claim 1, wherein the cutter guide is made of a clear material to allow the product underneath to be seen, minimizing risk of incorrect cutting of the product.

8. The apparatus of claim 1, wherein cutter guide is structured on an underside thereof to match structural features on the lower half to ensure that the cutter guide is locatable in the lower half only where it is impossible to cut a seed in the product.

9. The apparatus of claim 1, wherein cutter guide has one of a central groove and pocket running a length of the cutter guide to receive the cutting instrument to ensure cuts in the product by the cutting instrument are in a substantially straight line.

10. The apparatus of claim 1, wherein cutter guide has structural elements on an underside thereof that cover seeds of the product, minimizing a potential for interaction between the cutting instrument and the seeds and subsequent damage to the seeds.

11. The apparatus of claim 1, wherein the apparatus further comprises an outer shielding case that encases the upper and lower halves of the thermoformed tray.

12. The apparatus of claim 11, wherein the outer shielding case is telescoping case.

13. The apparatus of claim 11, wherein the outer shielding case is formed of at least one of lead, stainless steel, bismuth- or tungsten-loaded plastic, and pewter.

14. The apparatus of claim 11, wherein the outer shielding case is sealed with a shrink-wrap material.

15. The apparatus of claim 1, wherein at least one of the upper half and the lower half of the thermoformed tray is formed of a transparent material.
